(19) United States
(12) Patent Application Publication
(10) Pub. No.: US 2014/0373374 A1
(43) Pub. Date: Dec. 25, 2014

(54) DEVICE FOR FITTING AND DETERMINING THE SIZE OF A PATIENT INTERFACE

(71) Applicant: KONINKLIJKE PHILIPS N.V., EINDHOVEN (NL)

(72) Inventors: Dmitry Nikolayevich Znamenskiy, Eindhoven (NL); Ruud Vlutters, Eindhoven (NL); Octavian Soldea, Kiryat-Bialik (IL); Karl Catharina Van Bree, Eindhoven (NL); Peter Chi Fai Ho, Pittsburgh, PA (US)

(21) Appl. No.: 14/368,311

(22) PCT Filed: Dec. 21, 2012
(86) PCT No.: PCT/IB2012/057604

§ 371 Date: Jun. 24, 2014

Related U.S. Application Data
(60) Provisional application No. 61/582,499, filed on Jan. 3, 2012.

(51) Int. Cl. G01B 3/34; A61M 16/06

(52) U.S. Cl. CPC G01B 3/34; A61M 16/06; A61M 2016/0661
USPC 33/512

(57) ABSTRACT

The present invention relates to a device for fitting and determining the size of a patient interface, said device (10) comprising a sizing gauge(12), wherein the shape of the gauge (12) is configured to replicate the shape of a contacting surface of a patient interface with the portion of a patient's face, and a support (14) including a grip (16) for holding the device (10) by hand during use, which support (14) is mechanically coupled to said gauge (12).

### FIELD OF THE INVENTION

The present invention relates to a device for fitting and determining the size of a patient interface. The invention particularly relates to a device for fitting of patient interfaces, such as face masks for delivering gas to a patient.

### BACKGROUND OF THE INVENTION

More and more patients suffer from obstructive sleep apnea or obstructive sleep apnea syndrome (OSA). OSA is usually caused by an obstruction of the upper airway. It is characterized by repetitive pauses in breathing during sleep and is usually associated with a reduction in blood oxygen saturation. These pauses in breathing, called apneas, typically last 20 to 40 seconds. The obstruction of the upper airway is usually caused by reduced muscle tonus of the body that occurs during sleep. The human airway is composed of walls of soft tissue which can collapse and thereby obstruct breathing during sleep. Tongue tissue moves towards the back of the throat during sleep and thereby blocks the air passages. OSA is therefore commonly accompanied with snoring.

Different invasive and non-invasive treatments for OSA are known. One of the most powerful non-invasive treatments is the usage of CPAP (continuous positive airway pressure) or BiPAP (bi-positive airway pressure) in which a face mask is attached to a tube and a machine that blows pressurized air into the mask and through the airway in order to keep it open. Positive air pressure is thus provided to a patient through a hose connected to a patient interface, such as a face mask, that is worn by the patient. Usually, these face masks are worn using a head gear with straps that go around the back of the patient's head. An example of such a CPAP system is known from WO 2011/022779 A1.

Obviously, a correct fit of a patient interface (facial mask) on a user's face is of great importance to avoid gas leaks between the patient interface and the patient's face, and in order to serve for a good wearing comfort. Therefore, mask fitting and size determination is a great issue.

The selection of a CPAP mask with the proper mask geometry is one of the key factors determining the mask compliance and therefore the revenue of the mask producer. Fitting of a CPAP mask is a time and cost expensive procedure. First of all, the masks itself are expensive since they are individually fitted to the patient, respectively to the patient's face. Once fitted, they cannot be used on another person. Secondly, the fitting of the mask takes time which also enlarges the expenditure of time and therefore production costs. Thirdly, mask fitting is an unpleasant procedure for the patients.

Usually, sleep labs in which these patient interfaces are tested and individually fitted to the user have 10 to 20 differently sized and shaped testing masks from which 1 to 3 are usually selected for an actual fitting trial. The pre-selection of the shape and size of the mask is usually based on the patient's metadata (whether the patient is nose or mouth breather, or the earlier experience of the patient with CPAP masks, etc.). Another common way is the usage of simple sizing gauges.

In practice, two different types of CPAP sizing gauges are used in the described fitting procedure. The first known type of sizing gauges is a simple flat (two-dimensional) template gauge with cutouts that correspond to the different sizes of the mask perimeter. These flat gauges are held in front of the patient's face in order to roughly estimate the correct size.

However, due to their simplicity, these sizing gauges only allow to roughly estimate the correct mask size, but do not allow to estimate or predict the correct shape of the mask that optimally fits to the patient's face. These sizing gauges can, therefore, also not predict whether the fitting allows for unwanted air leakages or high pressure points.

The second type of sizing gauges available on the market comprises a bundle of silicon cushions that almost exactly correspond to the shape of the masks, each cushion for a different mask size. The cushions give an impression to the patient about the feeling of the actual mask. In other words, differently sized mask prototypes made of silicon are used as test masks for the fitting procedure.

However, these test masks have a number of serious limitations. First of all, these test masks are comparatively expensive. The costs of a test mask are almost comparable with the costs of a “real” mask. Secondly, a full face test mask could be perceived claustrophobic making the fitting procedure unpleasant and painful for the user. Thirdly, while the test mask gives a good impression about the feeling of the actual “real” mask, these large test masks obscure the visual inspection of the contour where the mask touches the face. A prediction of unwanted air leakages is thus also not or only hardly possible with these types of test masks.

Other types of fitting gauges which are, for example, used for the fitting of goggles are also not suitable to be used for the above-mentioned fitting of CPAP masks. FR 2 928 076 A1, for example, discloses a device that includes a reading unit to determine the correct size of an underwater goggle. Said reading unit is provided with size indicative information zones which are associated with a combination of marking zones of two markers, such that the correct size can be directly read from the size indicative information when the device is positioned on the face of the user. The reading of the size is carried out by determining the size indicative information zones corresponding to the combination of marking zones that mark respective positions of external and internal edges of the user's face. However, this approach seems to be far too complicated for the fitting of a CPAP mask, since this would require a completely parameterized shape concept of the CPAP mask that would make its production complicated and very cost intensive.

### SUMMARY OF THE INVENTION

It is an object of the present invention to provide a novel solution for a fitting device for patient interfaces, such as CPAP masks, which overcomes the above-mentioned disadvantages. In particular, it is an object to provide a low cost solution which can be easily cleaned and reused, is intuitively easy in operation, allows an optimized patient comfort during fitting, and enables a reliable visual inspection of the correct fitting. According to the present invention, this problem is solved with a fitting device of the kind mentioned initially, wherein said device comprises:
a sizing gauge, wherein the shape of the gauge is configured to replicate the shape of a contacting surface of a patient interface with the portion of a patient's face, and
a support including a grip for holding the device by hand during use, which support is mechanically coupled to said gauge.

The sizing gauge preferably has the same shape as the perimeter of the cushion that is attached to the patient interface (the CPAP mask) and used as contact element between the mask and the patient's face to seal the interface. The sizing gauge is thereto simply designed as a wire frame that preferably has a form of a ring which is adapted to the shape and contours around the mouth and nose of a patient's face.

The inner part of said sizing gauge is open and not filled with any material, so that the sizing gauge only represents the outer contact areas of a patient interface/mask of a corresponding size. In contrast to the usage of a closed test mask which is in practice usually used for fitting, the proposed fitting gauge only replicates the important contact area between the mask and the patient's face during use and, due to its open wire frame structure, does not fully cover the nose and mouth of the patient. Therefore, it serves to deliver the important information if the shapes and sizes of the corresponding patient interface fits on the patient's face, and at the same time allows the patient to breathe freely through the mouth and nose during the fitting procedure.

The simple sizing gauge is apart from that easier to produce and less cost intensive compared to the above-mentioned full test masks. Besides that, it is easier to clean and may therefore often be reused in a hygienic manner. Due to its open structure, preferably being designed as a ring that surrounds the face contours around the nose and mouth, it does not lead to a claustrophobic impression for the user and thus optimizes the patient's comfort during fitting. As mentioned the sizing gauge preferably resembles the form of a ring, but other forms such as an opening in the ring may also be possible.

Different sizes and shapes of the sizing gauges may be easily reused corresponding to the different sizes and shapes of the mask interfaces. During the fitting procedure, the correct size and shape of the CPAP mask may be easily determined by pressing differently sized and shaped sizing gauges into the patient's face until the correct size and shape is found that perfectly fits to the patient's individual face.

Thus, the “real” mask does not need to be produced in advance, before fitting, but may be produced afterwards according to the sizing gauge that has been found to fit the patient's face best during the fitting procedure. Sleeping labs therefore no longer need to store different kinds (different sizes and shapes) of expensive test masks, but only need to store different kinds (different sizes and shapes) of the herein proposed fitting devices.

The open wire frame structure of the sizing gauge furthermore allows an easy visual inspection of the correct fitting since gaps that might occur between the sizing gauge and the patient's face, which could lead to unwanted air gaps of the later produced mask, can be easily identified.

This easy visual inspection is not possible when using a complicated test mask that obscures major parts of the patient's face and complicates the visual inspection of the correct fit. Apart from that, the proposed sizing gauge also allows to identify pressure marks that might occur due to an incorrect fit. The sizing gauge may, for example, also comprise a visualizing material, such as ink, that is dispersed around the perimeter of the ring and produces an imprint on the patient's face in order to ease the visual inspection and see where the sizing gauge has a correct contact to the patient's face, and where not.

The proposed support that includes a grip for holding the device by hand includes the main advantage of allowing to press the gauge against the patient's face without touching it. This does not only simplify the handling of the device, but also enables a clean and hygienic fitting. Apart from that, direct touches and contacts of the fitting assistant or the physician with the patient's face, which might be uncomfortable and unpleasant for the patient, can be avoided. When applying the gauge, a sleep technician operator thus only has to push the sizing gauge to the patient's face by holding the fitting device at a single grip of the support.

According to an embodiment of the present invention, the sizing gauge is shaped three-dimensionally and adapted to the contour of a portion of a patient's face. Preferably, the sizing gauge is adapted to the shape of the patient's face around the nose and mouth parts, i.e. to the shape of the chin, the cheek and the area between the eyes (the nose bridge).

The fact that the sizing gauge is preferably realized as a three-dimensional ring in other words means that its shape is adapted to the three-dimensional contours of the patient's face and/or has a form that differs from a planar, two-dimensional form, i.e. includes parts that protrude from the planar form. A three-dimensional sizing gauge ring repeats the geometry of a specific cushion of the mask/patient interface when the mask is applied to an average face and preloaded with a certain pressure.

Thereto the shape of the sizing gauge preferably replicates the outer perimeter of a respiratory CPAP mask that is pressed against the patient's face. In other words, the proposed sizing gauge replicates the three-dimensional shape of the contacting (mating) surface of the cushion of the patient interface in situations where the patient interface (the CPAP mask) is pressed against the patient's face during use. Since the cushion of the patient interface is usually made of a flexible material, such as a silicon rubber, the cushion is at least partly compressed as soon as the CPAP mask is attached to and pressed onto the patient's face. The shape of the cushion (mask interface) therefore differs in the unloaded situation (in which the CPAP mask is not attached to the patient's face) from the shape of the cushion when the CPAP mask is attached to and pressed onto the patient's face. This means that for a realistic replication the sizing gauge needs to replicate the shape of the patient interface when being pressed onto the patient's face.

Some CPAP masks can be built to provide an optimal fit to a certain average three-dimensional face model. This three-dimensional face model could either be a “real” head sculpture or a three-dimensional computer model. In order to realize an appropriate sizing gauge for such CPAP masks, it is according to an embodiment of the present invention preferred that the shape of the sizing gauge follows a three-dimensional contour of a predetermined three-dimensional face model. By comparison of the face model and the sizing gauge or fitting the sizing gauge to the face model it can be easily proven if the sizing gauge is appropriately designed.

According to a still further embodiment, the sizing gauge is configured to encircle a face portion including the mouth and the nose of a patient, so that the mouth and the nose of the patient are not covered and protrude through the gauge when the gauge is pressed against the patient's face. The sizing gauge is thereto preferably configured to replicate the pressure distribution of the patient interface which is, during use, pressed against a portion of the patient's face. Thus, by comparing the pressure distribution of the sizing gauge and the pressure distribution of the patient interface it can be checked if the sizing gauge is appropriately designed.

Therefore, by holding the fitting device at the grip of the support and pressing it to the patient's face, a force is transmitted from the grip through the support to the sizing gauge such that the top and bottom part of the sizing gauge are pushed against the face with forces proportional to their distance from the grip of the support. In this way, the gauge self-positions on the patient's face creating a specific pressure distribution by only pressing it with the grip against the patient's face.

This means that a sleep technician operator only needs to hold the fitting device at a single point, i.e. at the grip, which can be done using only one hand or even only a few fingers. The above-mentioned pressure distribution that replicates the pressure distribution of a corresponding patient interface/mask during use can be easily computed from the position of the grip and the three-dimensional geometry of the sizing gauge. Thus, the desired replicated pressure distribution can be configured by correspondingly adapting the shape of the sizing gauge and the position of the support and its grip relative to the sizing gauge.

According to an embodiment, the center of the grip is thereto positioned at a predefined position with respect to the sizing gauge, in particular on a level with the geometrical center or the center of gravity of the sizing gauge, such that pressing the gauge against a patient's face almost exactly replicates the pressure distribution of the corresponding patient interface at the patient's face during use.

The sizing gauge is preferably made of a rigid material, while the support is preferably made of a flexible and/or bendable material. The support is thereto preferably realized as a (e.g. flexible and/or bendable) lever that sticks out from the sizing gauge, advantageously in perpendicular direction to the adjacent portion of the gauge. A flexible and bendable lever acts as a kind of flexible spring that bends as soon as the device is pressed to the patient's face while only holding the grip. This flexible spring nature of the lever allows soft touching of the face even though the sizing gauge is made of a rigid material. This, of course, improves the user's comfort.

The fact that the sizing gauge is preferably made of a rigid material allows for an easier and more reliable inspection of the correct fit, since a flexible gauge would deform too fast as soon as it is pressed against the patient's face. This would then corrupt the fitting.

The above-mentioned (e.g. flexible and bendable) lever is preferably on one end fixed to the sizing gauge at a single fixation point and has on the opposite second end a single punctual grip for holding the device by hand. The single connection from the lever to the sizing gauge at a single fixation point offers multiple benefits.

First of all, it reduces the pressure from the nose bridge as soon as the sizing gauge is pressed to the patient's face. In other words, it reduces the rigidity of the mechanical coupling between the top of the lever (the grip) where the fitting device is held by hand and the nose bridge onto which it is pressed. Secondly, a single connection between the lever and the sizing gauge realizes a very open and visible structure that does not induce an uncomfortable and claustrophobic feeling for the patient during the fitting process. Thirdly, the single connection and the flexible and bendable lever leads to a good self-positioning effect of the sizing gauge, meaning that the sizing gauge automatically self-positions itself to the correct position in the patient's face as soon as it is pressed against it. Lastly, such a single connection is easy to realize and thus simplifies the construction and minimizes the production costs.

According to a further embodiment of the present invention, the device includes a plurality of sizing gauges of different sizes, wherein each sizing gauge is configured to replicate the shape of a contacting surface of a correspondingly sized patient interface with the portion of the patient's face. Preferably, said plurality of differently sized sizing gauges are connected to each other, wherein each sizing gauge comprises a corresponding support including a grip for holding it by hand.

According to this embodiment, the fitting device in other words includes a number of differently sized and shaped sizing gauges which are connected to each other. In this case, a sleep technician operator may use a chain of differently sized sizing gauges and, during the fitting procedure, change between these sizing gauges in a fast manner. The technician may, for example, start with the largest sizing gauge, press it to the patient's face and, if it is too large, directly take the next smaller sizing gauge which is connected to the previous one in the manner of a chain. This allows speeding up the fitting procedure, since the sleep technician operator does not always need to change between different fitting devices or even search for the correct fitting device, since the fitting device already includes a number of sizing gauges.

The design of each sizing gauge within this sizing gauge chain can be realized in the same way as explained above, meaning that each sizing gauge is connected at a single point to a flexible and bendable lever which at its end comprises a grip for holding it and pressing it against the patient's face.

### BRIEF DESCRIPTION OF THE DRAWINGS

These and other aspects of the invention will be apparent from and elucidated with reference to the embodiment(s) described hereinafter. In the following drawings

FIG. 1 shows a first embodiment of the fitting device according to the present invention during use in a schematic way;

FIG. 2 schematically illustrates the technical principle of the first embodiment of the fitting device shown in FIG. 1;

FIG. 3 schematically shows the first embodiment of the fitting device in a side view;

FIG. 4 shows the first embodiment of the fitting device in a top view;

FIG. 5 shows a second embodiment of the fitting device according to the present invention in a perspective view; and

FIGS. 6A-D show sectional views of different types of cushions used as interfaces in CPAP masks in order to schematically illustrate the deformation of said cushions occurring when the CPAP mask is pressed against the patient's face.

### DETAILED DESCRIPTION OF THE DRAWINGS

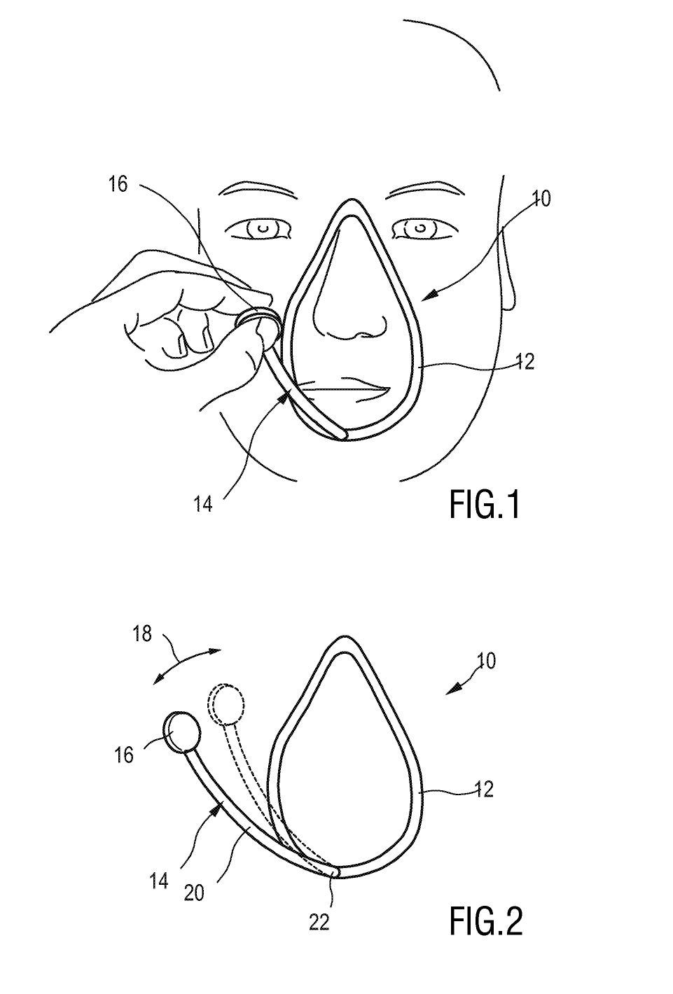

FIG. 1 shows a schematic, perspective view of an embodiment of the fitting and sizing device during use. The fitting and sizing device is therein in its entirety denoted with reference numeral 10. The device 10 comprises a sizing gauge 12 and a support 14 for holding the device 10 by hand during use. The sizing gauge 12 has the form of a ring that is adapted to the contours of a patient's face, i.e. adapted to the contours around the mouth, the cheeks and the area between the eyes (the nose bridge).

Said ring-shaped sizing gauge 12 replicates the shape of a respiratory mask, such as a respiratory mask that is used for CPAP. In particular, the ring-shaped sizing gauge 12 replicates the geometry of a cushion that is usually used in such CPAP masks as contact element/interface between the mask and the patient's face.

In contrast to “real” masks, the proposed sizing gauge 12 is realized as an open wire frame structure, meaning that it comprises a simple ring that is only filled with material at its outer perimeter while it is open (no material) in the inner part of the ring 12. While holding the sizing gauge 12 onto the patient's face in order to check the correct sizing and fitting, the mouth and nose of the patient is thus not covered, allowing the patient to breathe freely during the fitting procedure.

This open wire frame structure seems to be one of the main advantages in contrast to using a closed test mask that covers the patient's nose and mouth, and may be unpleasant for the patient, and could even lead to a claustrophobic anxiety state of the patient.

Apart from that, such a simple wire frame construction allows reducing costs and also increases the visibility of the interface between the gauge 12 and the patient's face, which again simplifies the fitting procedure since incorrect fittings and sizes can be visibly detected in a fast and easy manner.

The sizing gauge 12 may thereto, for example, be made of a single piece of plastic. This makes it easier to be washed and allows a re-usage for many patients. On the other hand, it is also disposable. In practice, it is thus no longer necessary to produce a set of very expensive test masks that have to be all produced for each patient individually, since it is now possible to test the fitting with the proposed cheap sizing gauges and only having to produce a single mask for each patient as soon as the correct geometry has been determined with the sizing gauge 12 that fits the patient best.

This means that only a set of cheap sizing gauges need to be produced for the mask fitting for each patient. In case the sizing gauge 12/the fitting device 10 is washable and may be reused, not even this is necessary, so that it suffices to have a set of fitting devices 10 on stock that may be used for all patients.

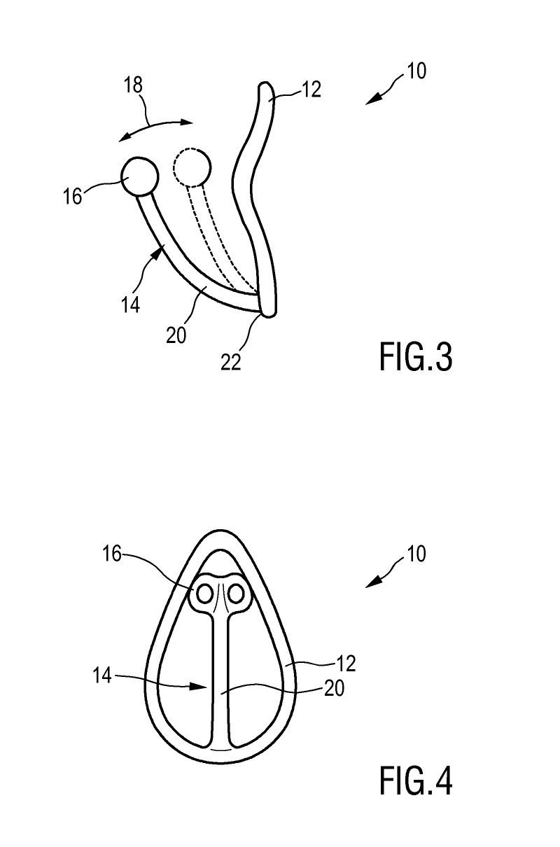

As it can be especially seen in the side view of FIG. 3, the sizing gauge 12 does not have a two-dimensional shape, but is shaped three-dimensionally. In contrast to two-dimensional sizing gauges, the proposed sizing gauge 12 is thus better adapted to the contours of the face of the patient, which again allows a more exact and realistic fitting. In case of a two-dimensional shape of the sizing gauge 12, the device 10 would only enable to roughly determine the correct size of the corresponding mask/patient interface, but not to determine the correct three-dimensional geometry of the cushion interface (contact element of the mask).

Additionally to the above-described sizing gauge 12, the proposed fitting and sizing device 10 furthermore comprises a support 14 which includes a grip 16 for holding the device 10 by hand during use. Said support 14 is mechanically coupled to the sizing gauge 12. It is preferably realized as a lever 20. This lever 20 is fixed to the ring-shaped sizing gauge 12 at a single fixation point 22 and comprises on its opposite side a single punctual grip 16. However, as this is exemplarily shown in the top view of FIG. 4, the grip may have variable shapes and does not necessarily need to have a punctual shape.

The lever 20 is preferably made of a flexible material, such as for example rubber. This allows the lever 20 to act like a flexible spring. As it can be schematically seen from FIG. 2, the lever 20 is bendable along a bending direction 18 which is preferably transversely oriented to the sizing gauge 12. In other words, this bending direction 18 may be substantially parallel to the normal direction of the main plain of the sizing gauge 12. Bending of the lever 20 occurs as soon as a force is applied to the grip 16. This bending effect allows using a rigid material for the sizing gauge 12 while still maintaining a good patient comfort. As soon as the sizing gauge 12 is pressed to the patient's face, the lever 20 bends and therefore dampens the pressure that is applied to the patient's face.

The rigid, three-dimensional sizing gauge ring 12 thereby repeats the geometry of the specific cushion to face interface replicating the situation of applying the correspondingly sized and shaped CPAP mask to the patient's face and preloading it with a certain pressure. The same pressure distribution is thus simulated with the proposed construction of the fitting device 10 including the flexible lever 20 and the rigid sizing gauge 12. In order to realistically simulate the pressure distribution of a real mask that has the same size and shape as the used sizing gauge 12, the shape of the three-dimensional sizing gauge 12 preferably corresponds to the perimeter of a normally loaded cushion. This means that one has to consider how the different types of CPAP mask and especially their cushions used as interface deform under the applied pressure that occurs as soon as the CPAP mask is attached to and pressed against the patient's face.

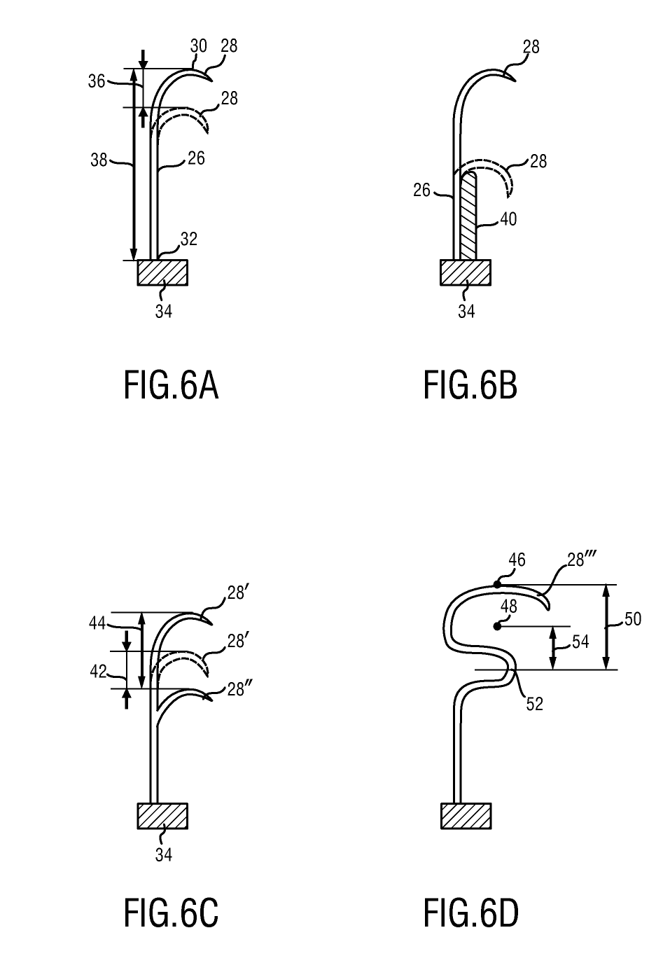

FIGS. 6A to 6D show sectional views of different types of existing cushions used as interfaces in CPAP masks in order to schematically illustrate the deformation of said cushions occurring when the CPAP mask is pressed against the patient's face. The illustrated cushions 26, which are in practice usually made of any kind of silicon rubber, commonly have a bent shape with a flap 28 at its highest point 30 which usually contacts the patient's face. These bent flaps 28 provide an additional sealing effect ensuring that gas leaks at the mask to face interface are avoided. At the lower end 32 the cushions 26 are usually connected to a so-called base plate 34 to which all remaining parts of the mask are connected (such as e.g. the air hose and the head gear for fixing the mask on the patient's head). Depending on the different types and shapes, the cushions 26 of course behave differently, meaning that they deform differently under the applied pressure that occurs as soon as the CPAP mask is attached to and pressed against the patient's face. For a single flap cushion 26 as illustrated in FIG. 6A, the cushion 26 usually deforms around 10% (indicated with reference numeral 36) of their total height 38 when being exposed to an average applied pressure. Cushions that are equipped with an additional gel cushion 40, as illustrated in FIG. 6B, usually deform to that extent that the flap 28 is deformed until it is being pressed against the gel cushion 40 if it is exposed to an average applied pressure that occurs when the mask is attached to the patient's face. A further known cushion design is the double flap design (see FIG. 6C), according to which the cushion 26 is equipped with two flaps 28′, 28″ arranged in parallel to each other. If these double flap cushions are pressed against the patient's face, the upper flap 28′ is usually deformed up to ⅔ of the distance between the two flaps 28′, 28″, i.e. the distance 42 in the deformed, loaded state is around ⅓ of the unloaded distance 44 between the two flaps 28′, 28″. A so-called flap grove cushion 26 as shown in FIG. 6D which has a curly shaped cushion 26 under an average applied pressure deforms to such an extent that the highest point 46 is shifted to point 48, wherein the distance between the top sealing flap 28′″ and the middle of the grove 52 is around ½ or less (indicated with 54) compared to the distance in the unloaded state (indicated with 50).

Bearing these different deformation behaviors in mind, it is possible to accurately design the shape of the sizing gauge 12 that realistically replicates the shape and pressure distribution of the real mask. This allows a realistic but still simple and low cost mask fitting.

An additional advantage of the proposed sizing and fitting device 10 is that the grip 16 allows holding the device 10/the gauge 12 with only a few fingers as this is schematically shown in FIG. 1. Therefore, when applying the gauge 12 to the patient's face, a force is transmitted through the lever 20 to the sizing gauge 12, such that the top end bottom part of the ring-shaped sizing gauge 12 is pushed to the patient's face with forces proportional to their shoulder distance from the top of the lever 20, i.e. from the grip 16.

Since the lever 20 is fixated on the sizing gauge 12 at a single fixation point 22, the sizing gauge 12 automatically self-positions on the patient's face creating a specific pressure distribution that realistically resembles the pressure distribution of a real mask. This pressure distribution can be easily computed from the position of the grip 16 with respect to the sizing gauge 12 and the three-dimensional geometry of the sizing gauge 12.

The single connection from the lever 20 to the sizing gauge 12 includes further additional advantages. On the one hand, it reduces the pressure that is applied to the nose bridge of the patient. On the other hand, it does not only simplify the construction and thus minimizes the costs, but also prevents a claustrophobic feeling of the patient.

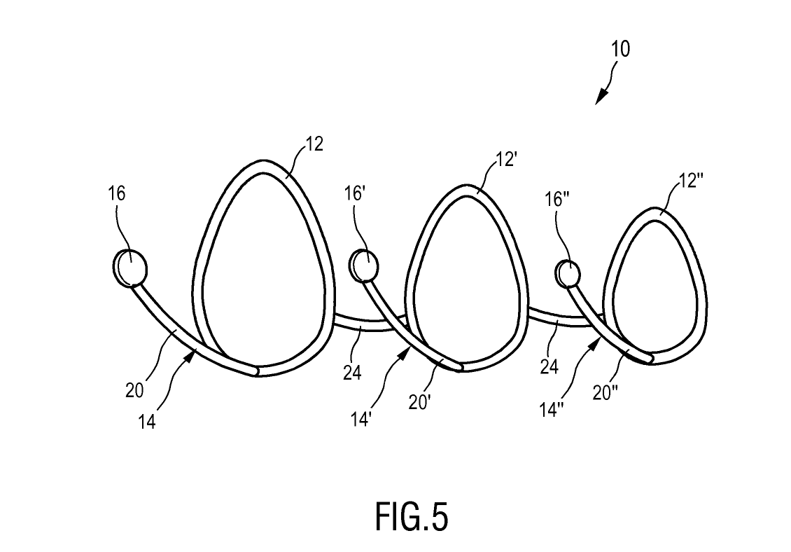

A second embodiment of the sizing and fitting device according to the present invention is shown in FIG. 5. In this embodiment, the device 10 includes a plurality of ring-shaped sizing gauges 12, 12′, 12″ of different sizes. As it has been explained before, each ring-shaped sizing gauge 12, 12′, 12″ is configured to replicate the shape of a mating surface of a correspondingly shaped and sized patient interface/CPAP mask with the portion of the patient's face. The plurality of differently sized and shaped sizing gauges 12, 12′, 12″ are connected to each other by a connection element 24. The connection element 24 may be realized in many ways. It may, for example, be realized by a chain or a simple piece of plastic that connects the different sizing gauges 12, 12′, 12″ with each other. Each ring-shaped sizing gauge 12, 12′, 12″ comprises a corresponding support 14, 14′, 14″ which includes a grip 16, 16′, 16″. These supports 14, 14′, 14″ and grips 16, 16′, 16″ are realized in the same way as explained with reference to the first embodiment above.

In this case, a sleep technician operator may use a chain of differently sized sizing gauges and, during the fitting procedure, change between these sizing gauges in a fast manner. The technician may, for example, start with the largest sizing gauge, press it to the patient's face and, if it is too large, directly take the next smaller sizing gauge which is connected to the previous one in the manner of a chain. This allows speeding up the fitting procedure, since the sleep technician operator does not always need to change between different fitting devices or even search for the correct fitting device, since the fitting device already includes a number of sizing gauges.

The design of each sizing gauge within this sizing gauge chain can be realized in the same way as explained above, meaning that each ring-shaped sizing gauge is connected at a single point to a flexible and bendable lever which at its end comprises a grip for holding it and pressing it against the patient's face.

In summary, the present invention proposes a disposable three-dimensional sizing gauge ring with a spring handle that may be used, in particular for CPAP mask fitting. The proposed device is a low cost device which can be easily cleaned and reused, if needed. The device is intuitively easy in application and serves to give an impression of the actual, “real” pressure distribution of a CPAP mask and reveals air gaps and high pressure points for quick visual inspection and mask selection. It furthermore improves the prevention of patient's claustrophobic reactions and optimizes the patient comfort making the fitting procedure more pleasant for the patient.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

Any reference signs in the claims should not be construed as limiting the scope.

1. A device for fitting and determining the size of a respiratory mask, said device comprising:
a ring-shaped sizing gauge, wherein the shape of the sizing gauge is configured to replicate the shape of a three-dimensional contacting surface of the p respiratory mask with a portion of a patient's face, and
a support (14) including a grip for holding the device by hand during use, which support is mechanically coupled to said gauge.

2-3. (canceled)

4. The device according to claim 1, wherein the shape of the sizing gauge replicates the outer perimeter of a respiratory mask, that is pressed against the patient's face.

5. The device according to claim 1, wherein the shape of the sizing gauge replicates a three-dimensional contour of a predetermined three-dimensional face model.

6. The device according to claim 1, wherein the sizing gauge is configured to encircle a face portion including the mouth and the nose of a patient, so that the mouth and the nose of the patient are not covered and protrude through the gauge when the gauge is pressed against the patient's face.

7. (canceled)

8. The device according to claim 1, wherein the centre of the grip is positioned at a predefined position with respect to the sizing gauge, in particular on a level with the geometrical centre or the centre of gravity, of the sizing gauge, such that pressing the gauge against a patient's face replicates the pressure distribution of the corresponding respiratory mask at the patient's face during use.

9. The device according to claim 1, wherein the sizing gauge is made of a rigid material and the support is made of a flexible, bendable material.

10. The device according to claim 1, wherein the support comprises a flexible lever.

11. The device according to claim 10, wherein said lever is bendable.

12. The device according to claim 10, wherein said lever sticks out from the sizing gauge.

13. The device according to claim 10, wherein said lever is on one end fixed to the sizing gauge at a single fixation point and has on the opposite second end a single punctual grip for holding the device by hand.

14. The device according to claim 1, wherein the device includes a plurality of sizing gauges of different sizes, wherein each sizing gauge is configured to replicate the shape of a contacting surface of a correspondingly sized respiratory mask with the portion of the patient's face.

15. The device according to claim 14, wherein the plurality of differently sized sizing gauges are connected to each other, and wherein each sizing gauge comprises a corresponding support including a grip for holding it by hand.
